(19) United States
(12) Patent Application Publication
(10) Pub. No.: US 2004/0006312 A1
(43) Pub. Date: Jan. 8, 2004

(54) Hypodermic syringes

(76) Inventors: Jeremy Francis Donnan, Balerno (GB); John Targell, Ayrshire (GB)

Correspondence Address: BAKER & DANIELS, 111 E. WAYNE STREET, SUITE 800, FORT WAYNE, IN, 46802

(21) Appl. No.: 10/615,226

(22) Filed: Jul. 8, 2003

Related U.S. Application Data
(63) Continuation-in-part of application No. 10/228,319, filed on Aug. 26, 2002, now abandoned.
Application No. 10/228,319 is a continuation of application No. 09/707,176, filed on Nov. 6, 2000, now Pat. No. 6,454,745.

(30) Foreign Application Priority Data
Nov. 10, 1999 (GB) GB 9926505.1
May 27, 2003 (GB) GB 0312036.7
Nov. 10, 1999 (GB) GB 9926505.0

(51) Int. Cl. A61M 5/00

(52) U.S. Cl. 604/181; 604/93.01

(57) ABSTRACT

The barrel of a hypodermic syringe is provided with a restrictor in the vicinity of its open end, the restrictor having being designed so as ordinarily to retain liquid within the barrel prior to fitting of a needle unit to the barrel but being adapted to allow passage of a needle and needle mounting hub therethrough.

### BACKGROUND OF THE INVENTION

This invention relates to the fitting of needle components to the barrel of a hypodermic syringe.

It is common practice to fit a syringe with a filling needle for the purpose of drawing liquid into the barrel of the syringe by retraction of its plunger from a vial often of the kind having a rubber cap which is penetrated by the filling needle. After the syringe has been charged, the filling needle is removed and replaced by a needle to be used for administration of an injection into a patient. Yet again it is often necessary to change a needle to meet patient requirements.

During the change of needle where the syringe is of a type having a large open end to permit needle retraction, care must be taken to avoid spillage of the contents of the syringe through the open end of the barrel.

It is an object of the present invention to provide a liquid retaining arrangement at the open end of a syringe barrel which overcomes the problem aforesaid.

### SUMMARY OF THE INVENTION

In one aspect of the present invention there is provided a restrictor or seal adapted to be fitted over the open end of the barrel of a hypodermic syringe comprising a cap of a non-latex elastomer having a central aperture which is sufficiently small as ordinarily to retain liquid therebehind but which can be deformably expanded to allow passage of a needle mounting portion for engagement with the barrel.

The cap may be of silicone rubber.

The aperture may be circular.

The aperture may take the form of a number of slits extending radially outward from the centre of the cap.

The cap may comprise a circular disc having a cylindrical skirt extending from its rear face.

The internal periphery of the skirt may be provided with axially spaced circumferential ribs adapted to engage with circumferential grooves on the end of the barrel to hold the cap in position.

The invention also includes a syringe barrel fitted with a seal as aforesaid.

The syringe barrel may be used with both a filling needle assembly and an injection needle assembly in which case the cap is expandable to allow fitting of both the filling needle and the injection needle to the barrel.

The arrangement may be such that the mounting portion of the injection needle is engaged with the barrel through a one-way coupling means formed by interfitting components on the needle mounting portion and the barrel whereby the injection needle once assembled to the barrel cannot be disengaged from the barrel (at least not without damaging the components). The filling needle may also be engageable with the barrel with the aid of the one-way coupling means associated with the barrel but in such a way that the filling needle may be subsequently disengaged.

The one way coupling means may comprise a threaded connection or a bayonet-type connection between a male part and a female part, one part being associated with the barrel and the other part being associated with the barrel.

The one way coupling means may for example comprise a threaded connection between a male and a female part, there being a void in an upstanding portion of the thread on each part and a barb in the void on one of the parts, the arrangement being such that when the parts are threadedly engaged, resilient deformation of at least one of the parts enables the barb to latch behind an end of a thread at the void in the other of the parts to prevent subsequent disconnection of the parts.

Another aspect of the invention concerns a threaded connection between a male part and a female part which, when properly threadedly engaged, cannot be disconnected, particularly, though by no means exclusively, suitable for joining components of fluid handling medical devices such as syringes, cannulas and catheters for example.

According to this aspect of the invention there is provided a threaded connection between a male and a female part, there being a void in an upstanding portion of the thread on each part and a barb in the void on one of the parts, the arrangement being such that when the parts are threadedly engaged, resilient deformation of at least one of the parts enables the barb to latch behind an end of a thread at the void in the other of the parts to prevent subsequent disconnection of the parts.

There may be on each part a two-start thread, the upstanding portion of each thread having a circumferential extent of less than 180° whereby there are voids between the ends of the two threads on each part and diametrically opposed barbs in the voids on the male part.

The height of each thread on the female part may increase from one end to the other which provides the abutment against which a barb latches.

More generally, the object of the present invention may also be achieved by providing the barrel of the syringe with a restrictor in the vicinity of its open end. In a further aspect of the present invention there is provided a barrel for use with a retractable needle assembly comprising a needle and a mounting hub for the needle, the barrel being provided with a restrictor located internally of the barrel at or adjacent an open end of the barrel, the restrictor having at least one aperture which is sufficiently small as ordinarily to retain liquid within the barrel and being adapted to allow passage of the needle and a mounting hub into the barrel.

In this manner, liquid introduced into the barrel, for example by means of a filling needle unit, may be prevented from “glugging” out of the barrel while the filling needle unit is being replaced by an injection needle unit, such egress of the liquid being restricted by virtue of surface tension effects associated with an aperture or apertures having appropriately small dimensions for the liquid involved.

In particular if, after uncoupling of the filling needle unit and before coupling of an injection needle unit of the retractable type, the barrel is handled normally, the risk of the liquid drug “glugging” out of the barrel is significantly reduced by the restrictor. Thus, for a water-based drug having, at a temperature of 20 degrees C., a viscosity of about 1 cP and a surface tension of about 73 dynes/cm, if the barrel is pointed downwardly, the restrictor will be capable of preventing liquid spillage at least as long the barrel is subjected to normal handling and is not shaken violently.

The aperture may take various forms and the term as used herein is to be construed as including a passageway along which the liquid can pass.

Typically the retractable needle unit comprises a needle, a mounting hub therefor, a restrainer for holding the hub in a forward position and a retraction mechanism operable, upon release of the hub from the restrainer, to drive the needle and mounting hub rearwardly from said forward position into the barrel.

The plunger may co-operate directly with the restrainer to effect release of the hub or alternatively at or near the end of its forward stroke of the plunger, the forward end of the plunger may contact the restrictor so that forward movement of the plunger is transmitted through the restrictor to the restrainer in order to effect release of the hub.

The liquids employed will usually have, at a temperature of 20 degrees C., a fluid viscosity in the range of 0.6 to 70 cP and a surface tension in the range of 20 to 100 dynes/cm.

The plunger is typically hollow and the retraction mechanism is operable to drive the needle into the hollow plunger.

The plunger may have a forward end which approaches said open end of the barrel during discharge of liquid from the barrel and may have at its forward end a part which closes said forward end but is severable or dislodgeable from said forward end to allow the needle and mounting hub to be driven by the retraction mechanism into the hollow plunger.

The restrictor may be a component separate from the barrel or it may be integral with the barrel.

The restrictor may be deformable, e.g. resiliently deformable, at least in the region of said aperture to allow passage of the needle and mounting hub therethrough.

The restrictor may include a sealing formation which co-operates with part of the needle unit whereby, during an injection stroke of the plunger, discharge of liquid from the unit is confined to the pathway provided by the needle.

Said aperture may comprise a single hole which may be circular or which may be provided with one or more inwardly directed projections for enhancing the ability of the restrictor to prevent egress of liquid from the barrel. For example, the periphery of said aperture may be of a castellated configuration formed by said projections.

The projection or projections may be capable of flexing to allow such passage of the needle and hub.

In some embodiments of the invention, said aperture of the restrictor may be sized so that the hub and needle can pass therethrough during needle retraction.

In other embodiments, said aperture may be of a size which is not sufficient to allow passage of the hub and needle. In such embodiments, the restrictor may include a portion which can be removed, e.g. broken away or dislodged, to allow passage of the needle and hub during the needle retraction process. Another alternative is for the restrictor to be capable of being disrupted, e.g. by virtue of being made of a brittle or frangible material, in such a way that it no longer blocks the needle and hub when needle retraction is required.

The restrictor may comprise an inner portion and an outer portion, the inner portion being severable or otherwise releasable from the outer portion to create an opening sufficiently large to allow passage of the needle and hub during needle retraction.

The inner and outer portions may be demarcated from one another by a zone or line of weakness (e.g. generally circular) at which the inner portion is severable from the outer portion during needle retraction. The zone or line of weakness may be provided by one or more webs of material interconnecting the two portions.

The inner and outer portions may alternatively be interconnected at a moulding interface therebetween such that the inner portion is severed or dislodged from the outer portion as the plunger approaches or reaches completion of its forward stroke.

The inner portion may be provided with said aperture.

The inner portion may be of tubular configuration defining a passageway constituting said aperture.

In one embodiment of the invention, the restrictor may comprise a disc located within the barrel.

When in situ within the barrel, the disc may be of generally conical configuration with an aperture at its “apex” and with its apertured “apex” located forwardly of the outer periphery of the disc.

The configuration of the disc, when in situ, may be such as to include an obtuse cone angle, typically of the order of 140 degrees.

The disc may be thin and flat when manufactured so that it can be pressed or cut out of strip or sheet material, injection or compression moulded or produced by other techniques so that the disc is not “handed” when manufactured but can take up the conical configuration.

Alternatively the disc may be manufactured with a generally conical configuration, e.g. by moulding.

The aperture in the disc may be a plain circular hole or it may be of other configuration. For example, its periphery may be provided with one or more fingers which will have the effect of reducing the apparent hole cross-section, thereby enhancing its ability to seal against liquid egress from the barrel.

The periphery of the aperture may be of castellated configuration, e.g. by virtue of one or more fingers as referred to above.

Where the aperture periphery is provided with a finger or fingers and/or a castellated configuration, the arrangement may be such that the finger or fingers or other such projections are capable of flexing to allow passage of the needle and hub.

The disc may be self-retaining in the barrel once inserted. Its outer periphery may if desired engage with a shoulder or within a groove in the wall of the barrel so that the disc is prevented from being dislodged.

The outer periphery of the disc may be provided with one or more fingers and/or be castellated to aid its deformation during insertion into the barrel and/or aid its retention once inserted into the barrel.

Typically the material of which the disc (or other form of restrictor) is composed may be a deformable polymeric material or an elastomeric material suitable for use in a medical syringe. The material may for instance be one which is suitable for exposure to gamma radiation and/or ethylene gas sterilisation.

The arrangement may be such that when the needle and hub assembly is fitted to the barrel, the rearward end of the needle/hub unit is located forwardly of the disc.

In another embodiment of the invention, the restrictor may be in the form of an annular flange projecting inwardly of the barrel and, optionally, rearwardly also, the flange defining an aperture which is sufficiently small as ordinarily to retain liquid within the barrel. This embodiment is particularly suitable for barrels which have a relatively low filling capacity, e.g. of the order of 1 cc.

The flange may be resiliently deflectable radially outwardly.

The flange may be formed integrally with the barrel adjacent its open end.

The flange may function also as a lip seal by co-operation with a component of the needle unit, e.g. by co-operation with a component which restrains the needle hub against retraction until the plunger approaches or reaches the end of its forward stroke.

In a further embodiment of the invention, the restrictor may be perforated or reticulated to prevent “glugging” of the liquid out of the barrel.

The restrictor may present an array of apertures distributed over the cross-sectional area of the barrel.

The restrictor may be of a frangible or brittle material or rendered frangible or brittle in a defined zone or zones thereof so that, during forward movement of the plunger, the integrity of the restrictor is disrupted to allow passage of the hub/needle assembly during needle retraction.

In yet another embodiment of the invention, the restrictor may comprise an inner part and an outer part which are normally coupled together but which are released from one another as the plunger approaches or reaches completion of its forward stroke.

The outer part may be restrained against forward movement or only allowed to move forwardly to a limited extent relative to the barrel.

After release of the inner part from the outer part, the inner part may leave an opening in the restrictor sufficiently large to allow passage of the needle/hub assembly during needle retraction.

The inner part may be provided with a central passageway for liquid flow into and out of the barrel, the passageway being sufficiently small as ordinarily to retain liquid within the barrel when pressure is not being applied to the plunger to move it forwardly.

The restrictor may be provided with an additional opening or openings of this nature in the outer part.

The two parts of the restrictor may be coupled together by moulding one part in the presence of the other part, e.g. by a two-shot moulding process, as disclosed in International Patent Application No. PCT/GB 02/01865, the entire contents of which are incorporated herein by this disclosure.

Alternatively the two parts may be coupled together via frangible sections of material which can readily break to allow release of the inner part from the outer part when required. For instance, the restrictor may be moulded from plastics material with thin webs of the plastics material interconnecting the two parts.

The hub and the restrainer may be formed, e.g. by a 2-shot moulding process, as plastics mouldings in such a way that the restrainer is axially captive with the hub, e.g. as disclosed in our prior International Patent Application No. PCT/GB 02/01865, the entire contents of which are incorporated herein by this reference.

The arrangement may be such that the injection needle unit is engaged with the barrel through a one-way coupling means formed by interfitting components on the needle unit and the barrel whereby the injection needle once assembled to the barrel cannot be disengaged from the barrrel (at least not without damaging the components).

The one way coupling means may comprise a threaded connection or a bayonet-type connection between a male part and a female part, one part being associated with the barrel and the other part being associated with the injection needle.

The one way coupling means may for example comprise a threaded connection between a male and a female part, there being a void in an upstanding portion of the thread on each part and a barb in the void on one of the parts, the arrangement being such that when the parts are threadedly engaged, resilient deformation of at least one of the parts enables the barb to latch behind an end of a thread at the void in the other of the parts to prevent subsequent disconnection of the parts. Such an arrangement is disclosed in our prior UK Patent No. 2353078, the entire disclosure of which is incorporated herein by this reference.

The coupling may be such that an internal thread section provided on the needle unit engages with an external thread on the barrel, or vice versa.

### DESCRIPTION OF THE DRAWINGS

The invention will be further apparent from the following description by way of example with reference to the figures of the accompanying drawing, in which:

FIG. 1 is a perspective view of the resiliently-deformable sealing cap associated with the barrel of a hypodermic syringe;

FIG. 2 is a fragmentary perspective view of a syringe barrel fitted with the cap and about to receive a filling needle device;

FIG. 3 is a view similar to that of FIG. 2 but wherein the barrel is about to receive the hub of an injection needle;

FIG. 4 is a perspective view of the forward end of a syringe barrel on an enlarged scale and with the resilient cap omitted; and

FIG. 5 is a perspective view on an enlarged scale of a connector arrangement for permanently coupling the injection needle to the syringe barrel.

FIG. 6 is a longitudinal sectional view showing the barrel of a hypodermic syringe fitted with a filling needle and sheath unit, the unit being provided with a restrictor in accordance with a first embodiment of the invention;

FIG. 7 is a view similar to FIG. 6 but showing the filling needle assembly removed from the barrel and the barrel filled with liquid;

FIG. 8 is a view similar to that of FIG. 7 but wherein the barrel is about to be fitted with an injection needle assembly;

FIG. 9 is an enlarged view showing the injection needle assembly fitted to the barrel;

FIG. 9A is a schematic view showing the periphery of the aperture formed in the restrictor;

FIG. 10 is a fragmentary perspective view in section of a syringe shown fitted with an injection needle of the retractable type, provided with a restrictor in accordance with a second embodiment of the invention;

FIG. 11 is a diagrammatic sectional view of a syringe showing a modification applicable to the embodiment of FIG. 10;

FIG. 12 is a fragmentary perspective view of another embodiment of the invention in which the restrictor is in the form of a frangible disc or the like;

FIG. 13 is a fragmentary perspective view of yet another embodiment in which the restrictor is of two part form allowing the central part to be produced with a passageway smaller than the lateral dimensions of the needle hub;

FIG. 14 is an enlarged perspective view of the restrictor of the embodiment illustrated in FIG. 13;

FIG. 15 is a fragmentary perspective view of a further embodiment in which the restrictor comprises an outer part and a central part; and

FIG. 16 is an enlarged perspective view of the restrictor of the embodiment illustrated in FIG. 15.

### DESCRIPTION OF EMBODIMENTS OF THE INVENTION

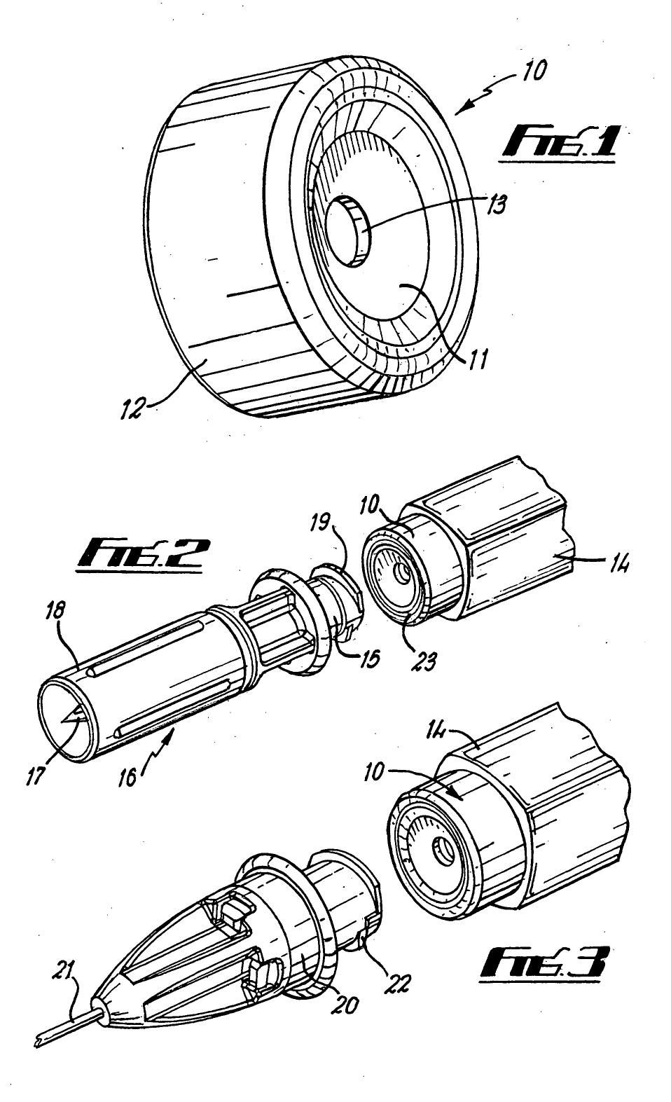

Referring firstly to FIG. 1, it will be seen that the seal, generally indicated at 10, comprises a cap having a circular disc portion 11 and a cylindrical skirt 12 extending from the rear face of the disc portion 11. A circular aperture 13 is provided through the centre of the disc portion 11. The seal 10 is a moulding of silicone rubber.

The inner periphery of the skirt 12 is provided with axially spaced circumferential ribs (not shown) which engage circumferential grooves on the forward end part of a syringe barrel 14 to enable the cap to fit securely over the open end of the barrel.

The aperture 13 is sufficiently small as to retain liquid therebehind having regard to the surface tension of the liquid even if the barrel 14 is pointed downwardly without deliberate violent movement such as shaking.

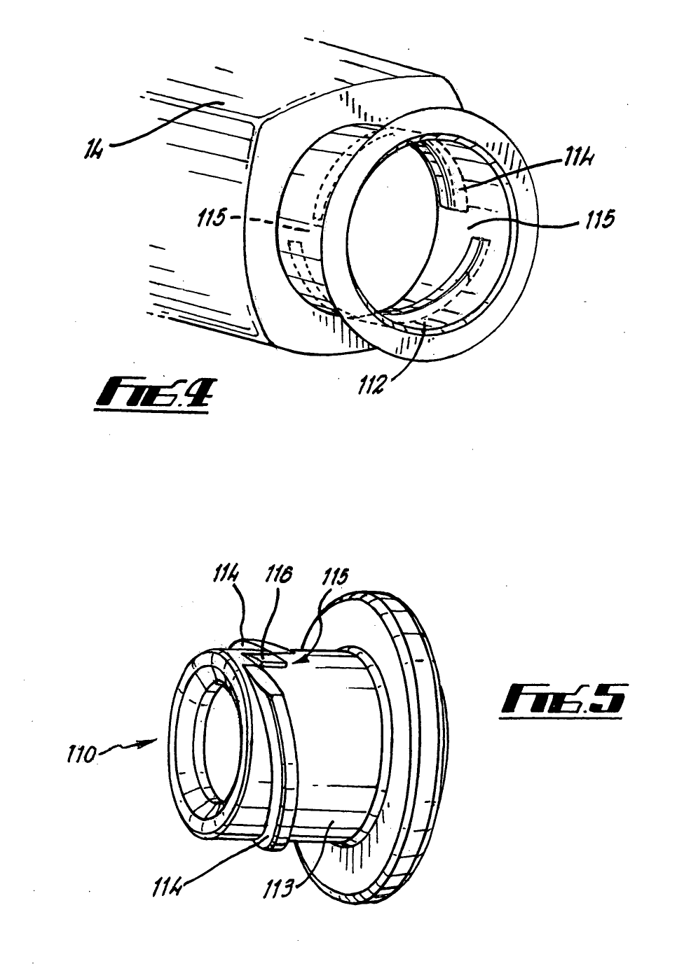

The aperture 13 may be resiliently expanded to permit entry of the mounting portion 15 of a filling needle assembly 16 whose needle 17 is protected until required by use by a frangible guard 18 (see FIG. 2). The rearward end 15 has a screw thread 19 which engages a complimentary thread (not shown) within the barrel 14 such that a raised sealing lip 23 of the seal is compressed between the mounting portion 15 and barrel 14 to form a tight seal.

After the barrel has been filled by insertion of the needle 17 into a vial of liquid which is drawn into the barrel 14 by retraction of the syringe plunger (not shown) the filling needle device 16 is disengaged and discarded and an injection needle 21 is fitted to the barrel. The hub or mounting portion 20 of the injection needle 21 also has a screw thread 22 which engages the thread within the barrel 14 again compressing the cap between the parts to form a seal.

During the needle change, i.e. during the interval between detachment of the filling needle device and fitting of the injection needle, the aperture 13 relaxes to its original size preventing accidental spillage of liquid from the barrel 14.

Referring now to FIGS. 4 and 5, a one way coupling means in the form of a threaded connection is illustrated. The coupling comprises a threaded female socket part 112 at the forward end of the barrel 14 adapted to receive a threaded male part 113 of a mounting portion 110 of the needle assembly. Each of the parts 112 and 113 is provided with a two-start right handed thread. The upstanding portion 114 of each of the four threads has a circumferential extent of less than 180° whereby there are voids 115 between the ends of the two threads on each part. On the part 113 are diametrically opposed barbs 116 in the voids 115.

When the parts are threadedly connected the barbs 116 are introduced to the voids 115 on the part 112 before relatively rotating the two parts. During rotation the part 112 which is of a plastics material resiliently deforms as the barbs 116 override the portions 114 of the threads on the part 112 before the barbs latch behind the ends of the threads on the part 112 to lock the parts 112 and 113 against disconnection. In this manner, the injection needle assembly is permanently coupled to the barrel and cannot be uncoupled without damaging the coupling connector 110.

It will be understood that the seal 10 will be present during fitting of the needle assembly to the barrel, i.e. in the course of engaging the male part 113 to the female part 112, the male part 113 will be passed through the aperture 13 in the seal 10 with consequent expansion of the aperture 13 to accommodate the male part.

The height of each thread on the female part increases from one end to the other which provides the abutment against which the barbs 116 latch to facilitate the threading operation and deformation of the part 112.

Whilst the injection needle assembly may be fitted to the barrel in a permanent fashion as described above, the filling needle assembly is releasably connected to the barrel so that it can be used in filling the barrel and then removed and replaced by the injection needle assembly. Fitting of the filling needle assembly may be through a coupling means 110 similar to that illustrated in FIGS. 4 and 5 except for the omission of the locking barbs 116. In other words, the part 112 of the barrel will be utilised and the mounting portion of the filling needle assembly will have the configuration of part 113 except for the omission of the barbs 116 thereby allowing the filling needle assembly to fitted to the barrel through the sealing cap 10 and subsequently disengaged following filling of the syringe barrel.

Although in FIGS. 4 and 5, the female part 112 is shown as being associated with the barrel and the male part 113 with the needle assembly, the arrangement may be reversed so that the male part is associated with the barrel and the female part with the injection or filling needle assembly.

The embodiments of FIGS. 6 to 16 are particularly suited for use with hypodermic syringes fitted with a needle unit of the retractable type, e.g. as described in for example our prior International Patent Application No. PCT/GB 02/01865. Typically, in such a syringe, the plunger has a forward end which approaches the open end of the barrel during discharge of liquid from the barrel and has at its forward end a part which can be severed or dislodged from the forward end of the plunger. The needle unit comprises a needle, a mounting hub therefor, a restrainer for holding the hub in a forward position and a retraction mechanism operable, upon release of the hub from the restrainer, to drive the needle and mounting hub rearwardly from said forward position into the hollow plunger. The severable part is severed from the forward end of the plunger prior to or during operation of the retraction mechanism. Release of the hub from the restrainer is effected by co-operation of the forward end of the plunger with the restrainer whereby forward movement of the plunger at or near the end of the forward stroke thereof is effective to displace the restrainer and effect release of the hub and the needle.

Referring to FIGS. 6 to 9 of the drawings, the tubular barrel 210 of a syringe is open at its forward end 212 and is fitted with a restrictor in the form of a non-latex, resiliently deformable disc or washer 214 having a central opening 216. In its relaxed state, the washer 214 may be flat having a diameter which is slightly greater than the internal diameter of the barrel at its forward end 212. When the washer 214 is pressed into the interior of the barrel, it takes up a slightly conical form by virtue of its diameter being slightly greater than the barrel internal diameter. This facilitates securing the washer in place at the forward end of the barrel. Instead of the washer being initially flat, it may instead be manufactured with a conical configuration. The periphery of the washer 214 contacts the internal wall of the barrel 210 and because of its conical configuration resists displacement in the rearward direction during filling of the barrel.

It will be understood that the washer need not be of conical configuration as long as it is secured in such a way that it is not displaced rearwardly during filling of the barrel. To facilitate location of the washer (whether conical or not), the barrel may be provided with an internal groove or shoulder 218 in or against which the washer 214 seats. When the washer is in situ in the barrel, the opening 216 is presented forwardly of the outer periphery of the washer. Although not shown, the forward end of the barrel may have a conical end formation located forwardly of the washer such that the washer, when inserted, takes up the form of the forward end of the barrel.

In FIG. 6, the barrel is shown fitted with a unit comprising a filling needle 220 and a protective sheath 222, the unit being coupled to the forward end of the barrel by a screwthreaded connection between the rearward end portion 224 of the unit 220, 222 and a forwardly projecting collar 226 of the barrel. The sheath 222 is designed to be broken off when the user is ready to use the filling needle so that the needle 220 can be inserted into a vial or other source of fluid which is to be drawn into the barrel in the usual way (involving retraction of a plunger, not shown, rearwardly along the barrel). As the fluid is drawn into the barrel, it flows directly through the opening 216 at the centre of the washer 214 and into the evacuated volume of the barrel.

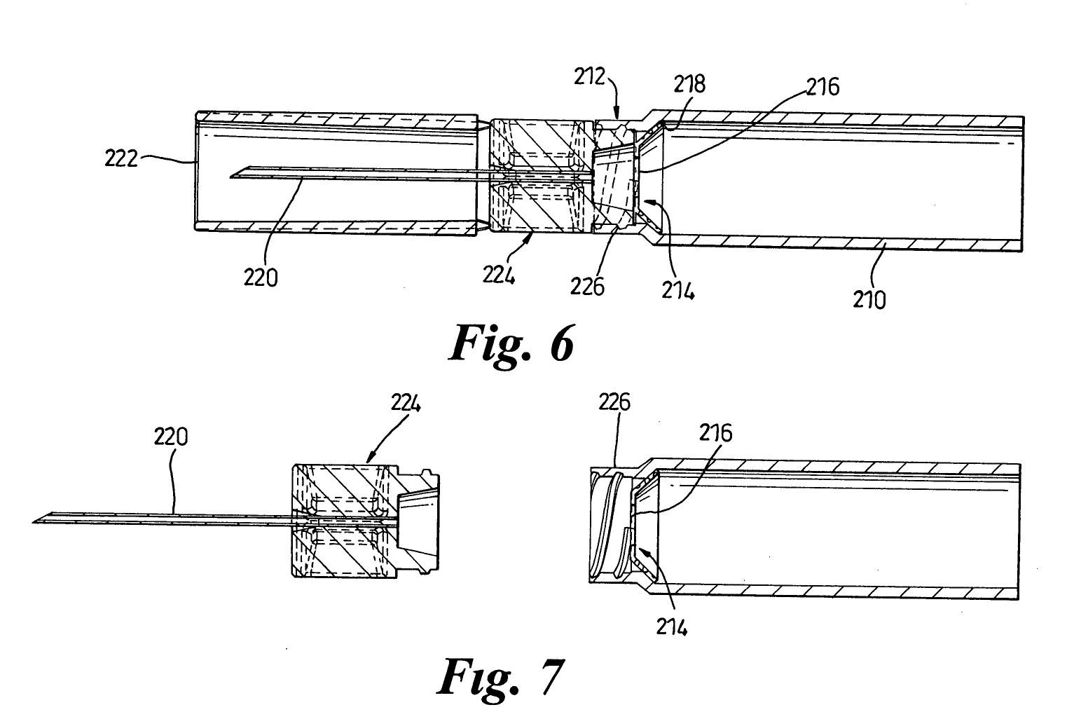

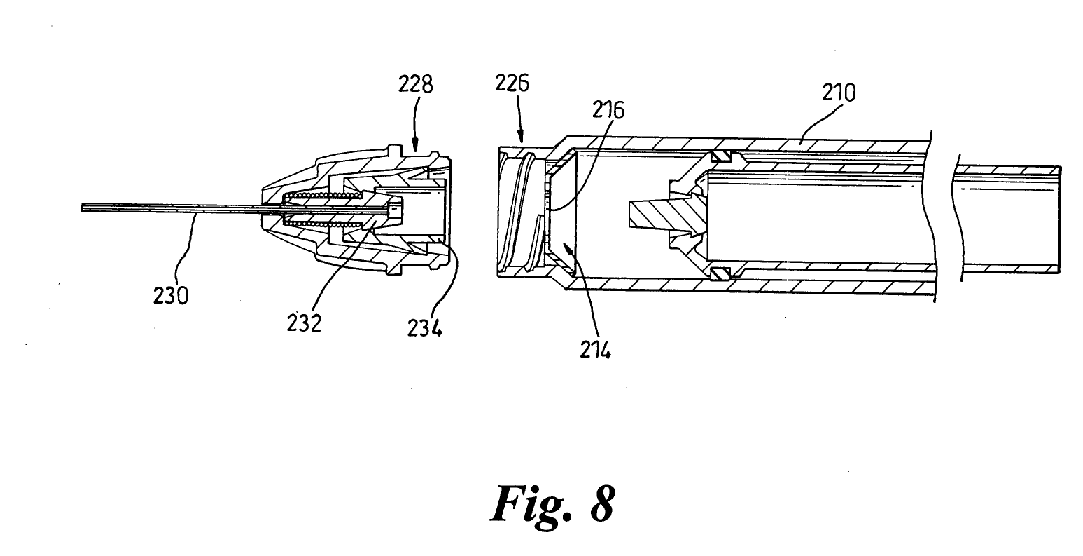

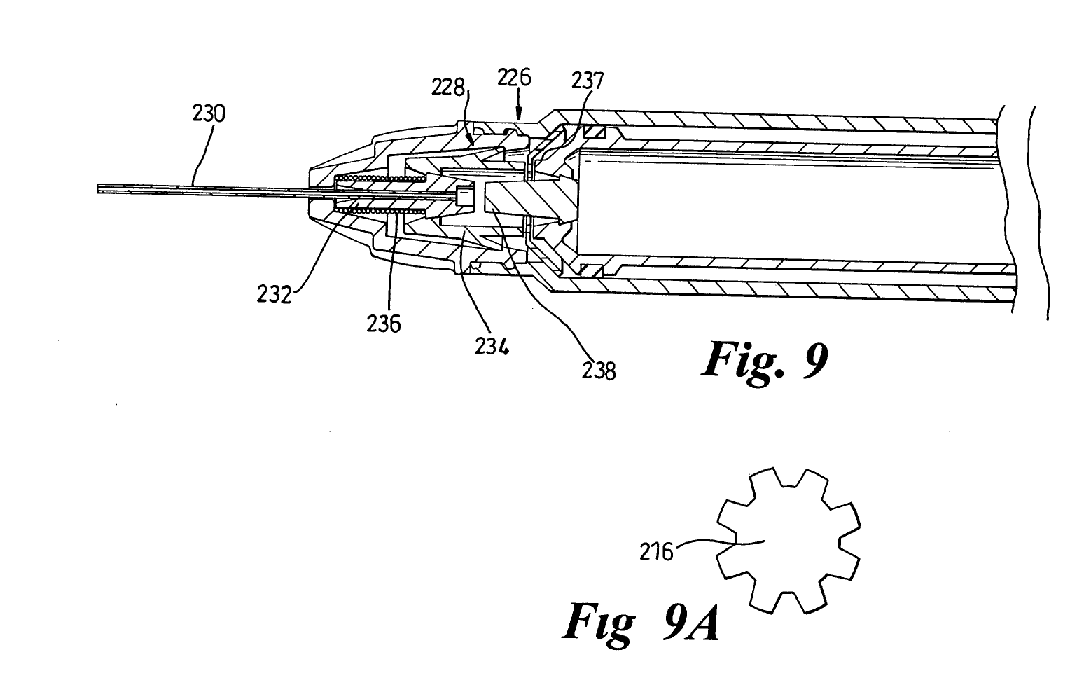

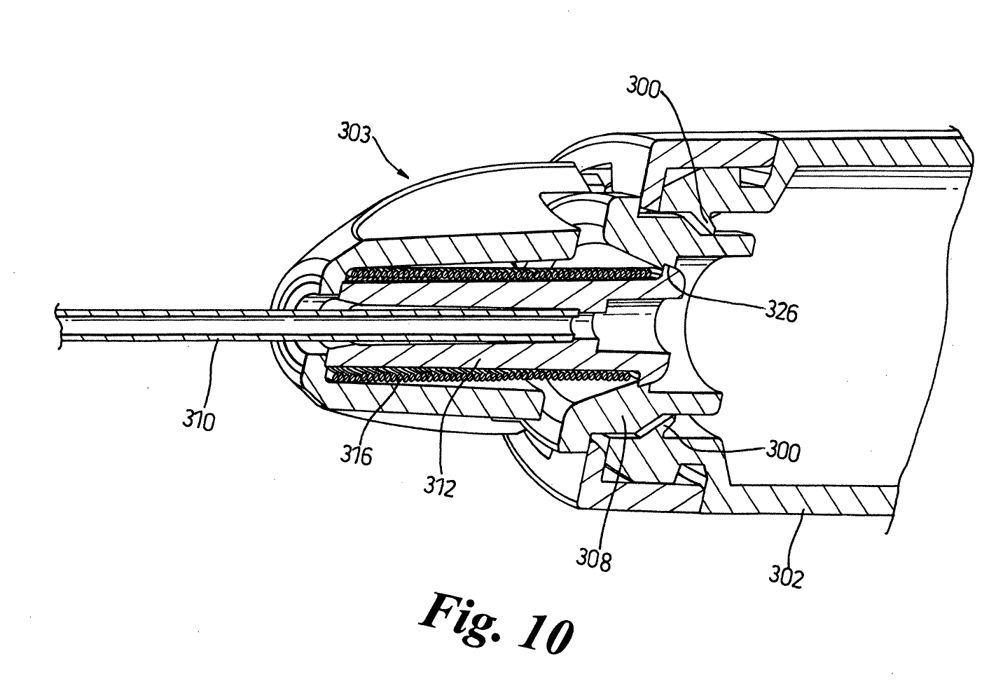

At this point, it is necessary to replace the filling needle unit with the injection needle unit. The filling needle is decoupled from the barrel 210 (see FIG. 7) and the injection needle unit is then fitted to the barrel (see FIGS. 8 and 9). If now the barrel is handled normally, e.g. inverted or shaken (but not unduly violently), the risk of the liquid drug “glugging” out of the barrel is significantly reduced by virtue of the washer 214 since the opening 216 forms a small hole at the base of a liquid filled chamber, namely the barrel, and is designed so that, because of the surface tension properties of the liquid, it cannot allow air in and liquid out at the same time, with the consequence that there is no flow either way. In this way, during interchange of needles, the liquid is retained in the barrel by the washer 214 provided that the barrel is not subjected to any unduly violent shaking or handling.

The injection needle unit comprises a mounting portion 228 for coupling to the barrel, an injection needle 230 carried by a hub 232 and a restrainer constituted by a crown 234. The hub 32 and needle 230 are biased rearwardly by a spring 236 for retraction at the appropriate time into the barrel and associated hollow plunger within the plunger. As disclosed in prior International Patent Application No. PCT/GB 02/01865, the hub 232 and crown 234 may be formed by a 2-shot moulding process so that they are captive with each other until the crown is displaced by collar 237 (see FIG. 9) of the plunger so to decouple the hub 232 and crown 234 and release the hub and needle for spring-driven travel into the barrel and plunger. It will be noted that, during the needle release operation, forward motion of the collar 237 is transmitted to the crown 234 via the washer 214 which is resiliently deformable. However, it will be understood that the present invention is not limited to triggering of the needle retraction mechanism in the manner just described; other methods known in the art may be used for retaining the hub and needle and releasing the same when needle retraction is required.

Prior to, during or just after release of the hub from the crown, the leading end 238 of the plunger will also be separated from the plunger and be driven into the interior of the plunger. In this context, the leading end of the plunger may, but need not necessarily, be produced as a 2 shot moulding as disclosed in International Patent Application No. GB00/04573, the entire disclosure of which is incorporated herein by this reference.

The gap between the forward end of the washer 214 and the rearward end of the crown is minimised to reduce drug wastage. It will be noted that the dimension of the hub is such that the hub and also the spring 236 may pass freely through the opening 216 in the washer once needle retraction has been initiated as a result of contact between the trailing end part of the crown 234 and the leading end 238 of the plunger.

The coupling between the mounting portion of the injection needle unit and the barrel is conveniently a one-way coupling as referred to above and may be implemented for example in the manner disclosed in UK Patent No. 2353078. Fitting of the filling needle assembly may be through a similar coupling means except for the omission of the locking barbs so that, in contrast with the injection needle unit, the filling needle unit may be readily removed when finished with.

In the embodiment of FIGS. 6 to 9, the opening 216 in the disc is circular; however, it may be of other configuration consistent with making use of surface tension effects to prevent the liquid “glugging” out of the barrel. For instance, the opening 216 may be of castellated configuration as illustrated in FIG. 9A so as to reduce the cross-sectional area of thereof, the projections extending radially inwardly but being readily deflectable so that they do not obstruct passage of the needle/hub assembly after needle retraction has been triggered.

Referring now to FIG. 10, in this embodiment the restrictor is constituted by an annular projection or flange 300 provided internally of the forward end of the barrel 302. The restrictor in this case may be formed integrally, as by plastics moulding, with the barrel. As shown in FIG. 10, the syringe is shown in a configuration in which the injection needle has been fitted following filling of the barrel with filling needle unit and removal of the latter. The hub 312 and crown 308 in this embodiment are coupled together at the moulding interface 326 by two-shot moulding in such a way that the parts are mechanically connected and possibly also fused or adhered together at the interface. Such an arrangement may also be employed in other embodiments of the invention illustrated herein.

Prior to fitting of the injection needle unit 303 and its sheath (not shown), it will be understood that the restrictor 300 will be effective to prevent spillage of liquid from the filled barrel provided that the diameter of the restrictor is sufficiently small. When the invention is implemented in this manner, the dimension of the opening in the restrictor 300 is determined by the need to allow passage of the assembly comprising the needle 310 and hub 312 during operation of the needle retraction mechanism. For this reason, this implementation of the invention is primarily intended for low capacity and hence small diameter syringes, e.g. syringes having a filling capacity of the order of 1 ml. In this case, the dimensions of the various parts can be made smaller, consistent with securing a restrictor hole sufficiently small that, for liquids in the viscosity range typically encountered for liquids to be administered by injection, the risk of egress of liquid from the barrel is prevented in normal use. As previously mentioned, typical properties for liquids to be used with syringes in accordance with the invention are a fluid viscosity in the range of 0.6 to 70 cP (typically about 1 cP) and a surface tension in the range of 20 to 100 dynes/cm (typically about 73 dynes/cm).

The restrictor 310 in the embodiment of FIG. 10 serves the additional function of providing a seal between the crown 308 and the internal wall surface of the barrel, being in the form of a resiliently deflectable lip seal so arranged that, during the forward stroke of the plunger (not shown), the lip seal extends inwardly and rearwardly so that it tends to deflect radially inwardly due to the pressure exerted thereby enhancing the sealing action. It will be seen that, when the crown 308 is in place and engages with the lip seal/restrictor 300, it deflects the lip seal/restrictor 300 radially outwardly. When the crown 308 is not present, the lip seal/restrictor 300 is in a relaxed undeflected condition in which the central hole is of reduced diameter compared with the diameter it expands to when the lip seal is deflected outwardly by the crown 308.

Operation of the syringe of FIG. 10 is generally similar to that described with reference to FIGS. 8 and 9 above and also to the syringe disclosed International Patent Application No. PCT/GB 02/01865 to which reference should be made for a more detailed description. After the barrel has been filled and the filling needle unit has been removed, the lip seal/restrictor 300 serves to retain the contents of the barrel awaiting fitting of the injection needle unit. Once the injection needle unit has been fitted, immediately prior to administering an injection, the needle sheath (not shown) is removed. Following insertion of the needle into the patient and operation of the plunger to force the contents of the barrel through the needle 310, the plunger eventually contacts and displaces the crown 308 forwardly to release the needle 310 and hub 312. At this time, the blocking portion 314 of the plunger 306 is also released to allow retraction of the needle and hub assembly into the hollow plunger, such retraction being effected by spring 316.

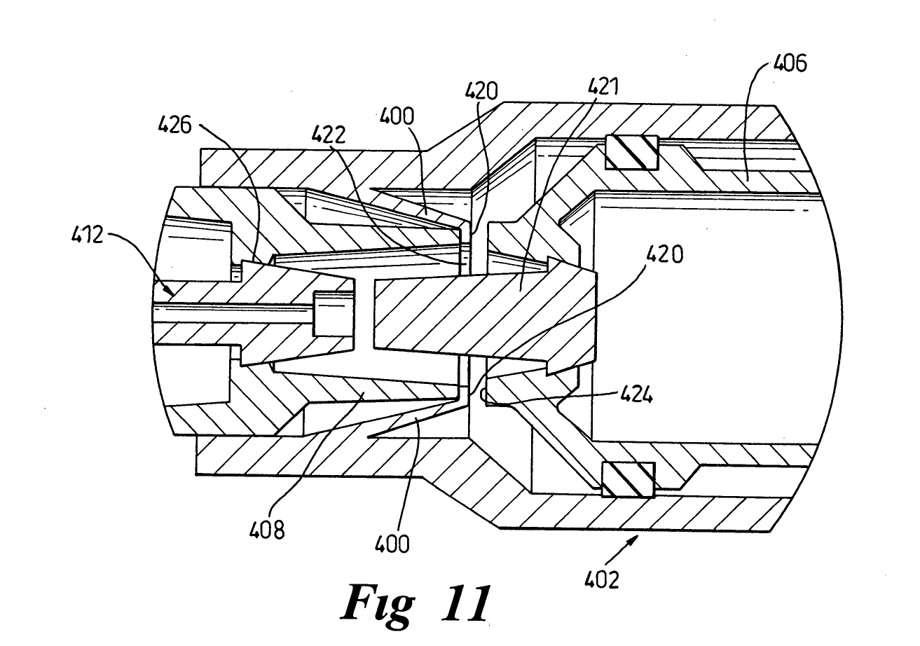

FIG. 11 illustrates a modification of the embodiment of FIG. 10 in which the restrictor 400 may again function as a resiliently deflectable lip seal but, at its inner periphery, is provided with an inwardly directed portion 420 which defines the aperture 422 through which the assembly of hub 412 and needle (not illustrated) of the injection needle unit can pass during needle retraction. The portion 420 may be integrally formed with the restrictor 400 which, in turn, may be integral with the barrel 402. The portion 420 may define a central circular aperture or alternatively the central aperture may be of other configuration, e.g. the portion 420 may be of castellated configuration or other configuration in which one or more parts project radially inwardly to a greater extent than other parts.

After the barrel 402 has been filled by means of the filling needle unit, it will be seen that egress of the liquid is restricted by the aperture 422. In the embodiment of FIG. 11, the central aperture 422 may be somewhat smaller than its counterpart in FIG. 5 thereby permitting use of the restrictor 400 of FIG. 11 with syringes over a wider range of filling capacities. During the forward stroke of the plunger 406, the forward end 424 of the plunger eventually contacts the portion 420 and continued pressure on the plunger is accompanied by displacement of the crown 408 and deflection of the restrictor 400 until needle retraction is triggered (by release of the hub from the crown at the interface 426) and the blocking portion 421 of the plunger is released, whereupon the hub and needle assembly is projected by the spring into the hollow plunger 406. Release of the blocking portion may occur just prior to, substantially simultaneously with, or just after release of the hub from the crown.

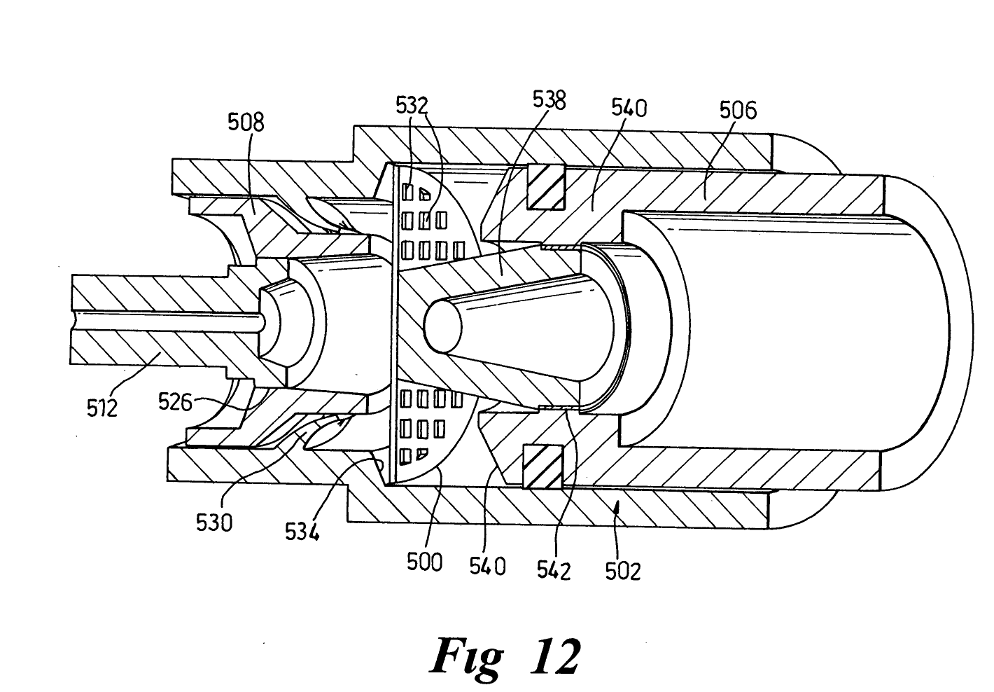

FIG. 12 illustrates an embodiment in which the barrel 502 is provided with a lip seal 530 as in FIGS. 10 and 11 but, in this case, the lip seal need not function as a restrictor. In this embodiment, a separate restrictor 500 is provided which is perforated, reticulated or otherwise formed with openings 532 which allow admission of liquid into the barrel 502 during filling but which, by virtue of surface tension effects, prevent or reduce the risk of liquid egress from the barrel when the filling unit is removed. The restrictor 500 may be in the form of a perforated or reticulated disc located within the barrel and, at its outer periphery, is blocked against forward movement, e.g. by internal shoulder 534 of the barrel.

The restrictor 500 is adapted to co-operate with the plunger 506 in such a way that, as the plunger approaches completion of its forward stroke, an opening is created in the restrictor 500 which is sufficient to allow the assembly of hub 512 and needle (not shown) to travel beyond the restrictor location. Such rearward travel of the hub and needle assembly can occur after the coupling at interface 526 is disrupted to allow hub release from the crown and the hub/needle assembly can then be driven into the hollow plunger 506 via the opening created by release of the blocking portion 538 from the rim portion 540 (at interface 542) as the plunger approaches or reaches completion of its forward stroke.

To allow the hub/needle assembly to pass through the restrictor, the restrictor 500 may be frangible so that it is broken by the forward advance of the plunger 506. For example, the restrictor 500 may be fabricated from a frangible or brittle material which will readily break or it may be formed with one or more lines or zones of weakness to allow part to be broken away to form the desired hole for passage of the needle and hub assembly.

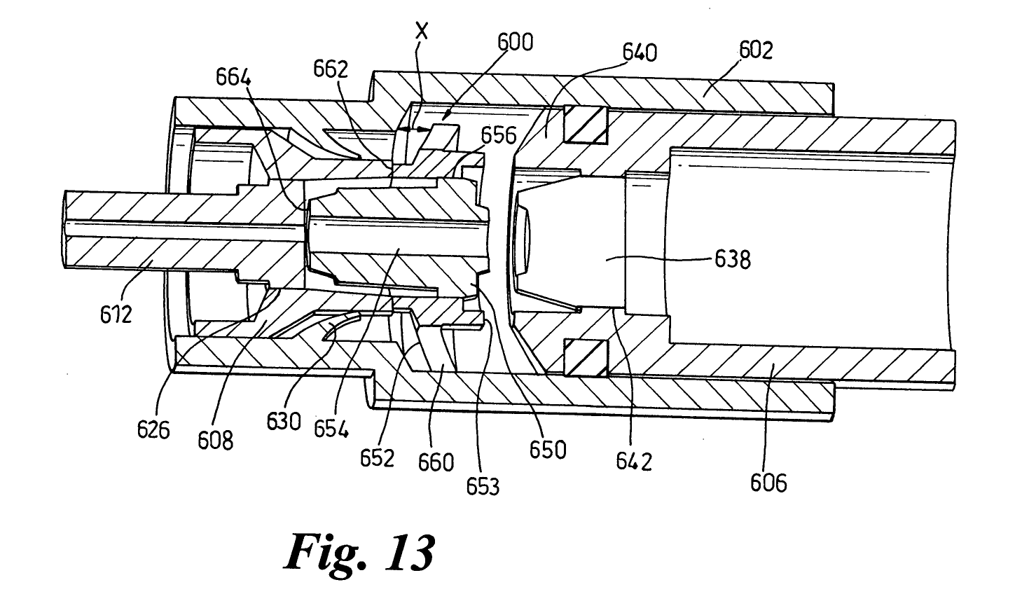

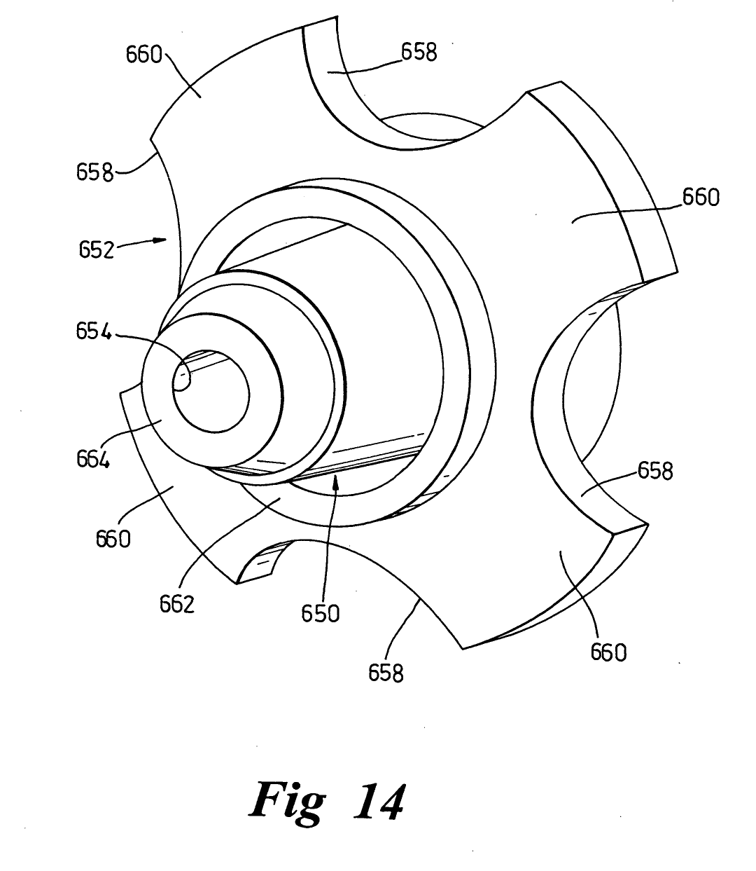

Referring to FIGS. 13 and 14, in this embodiment the restrictor 600 comprises a central part 650 and an outer part 652 which are coupled together in such a way that, at a suitable point during the forward stroke of the plunger 606, the central part 650 is freed from the outer part 652 so that the assembly of hub 612 and needle (not shown) can be driven by the spring (not shown) into the hollow plunger 606 along with the central part 650. The central part 650 is provided with a passageway 654 through which liquid can pass during the filling and injection procedures. This passageway is suitably dimensioned so that the liquid filling the barrel 602 is retained within the barrel by surface tension effects during the time between removal of the filling needle unit and fitting of the injection needle unit. Typically the diameter of the passageway 654 may be of the same order as conventional Luer fittings for syringes—e.g. of the order of 2.0 mm. As in the embodiments of FIGS. 10 to 12, the barrel may be provided with a lip seal 630 for co-operation with the crown 608.

The coupling between the parts 650 and 652 may be secured by producing the restrictor 600 by two-shot moulding of one or more plastics materials such that they are engaged with each other at the moulding interface 656, e.g. in the manner disclosed for the hub and crown in International Patent Application No. PCT/GB 02/01865. As previously mentioned, the coupling at the interface may be of a mechanical nature by contouring of the parts at the interface and/or it may involve fusion or adherence of the materials at the interface. However, it is to be understood that coupling arrangements, other than those created by two-shot moulding, may be used instead. The outer part may be of conical configuration, or adopt such a configuration when inserted into the barrel, with its outer periphery contacting the inner wall of the barrel 602 so that the restrictor 600 is readily movable in the forward direction but resists movement in the rearward direction.

In addition to the passageway 654, the outer part 650 may be formed with one or more openings in the form of for example apertures or cut-away portions 658 to provide further inlets for admission of the liquid during filling of the barrel. Such openings 658 will be dimensioned to prevent, by surface tension effects, liquid “glugging” out of the barrel 602. Of course, when the plunger 606 is pressed forwardly during the injection procedure, the liquid may exit via the openings 658 as well as through the passageway 654. By forming the openings 658 as cutaway portions as illustrated in FIG. 14, radially projecting arms 660 are formed which impart additional flexibility to the outer part 652 so as to allow it to be readily moved in the forward direction while resisting rearward movement.

The restrictor 600 in located within the barrel 602 with a small amount of clearance X. When the plunger 606 approaches completion of its forward stroke during the injection procedure, the leading end of the rim portion 640 initially contacts collar 653 of the outer part 652 while the blocking portion or nose 638 contacts the central part 650. Continued forward movement of the plunger is then transmitted via leading end 662 of the outer part 652 to the crown 608 and brings the leading portion 664 of blocking portion 638 into abutment with the trailing end of the hub 612. The various parts are designed so that, in the process of taking up the clearance X, the central part 650 is released from outer part 652 of the restrictor, the hub 612 is released from the crown 608 and the blocking portion 638 is released from the rim portion 640. Once these conditions are secured, the hub and needle assembly can be driven by the spring (not shown) into the hollow plunger 606 along with the central portion 650 of the restrictor 600.

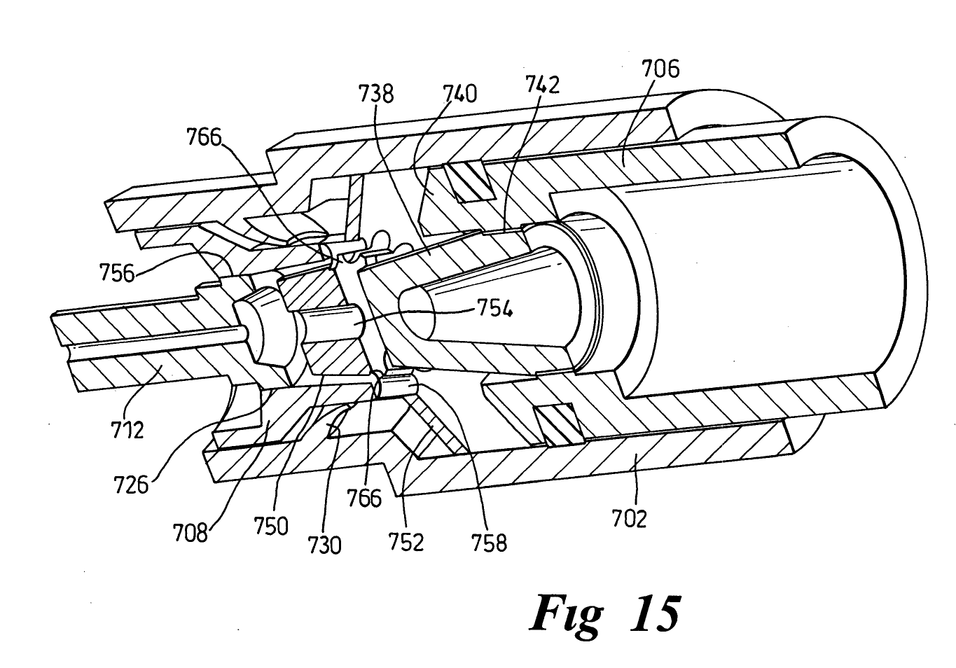

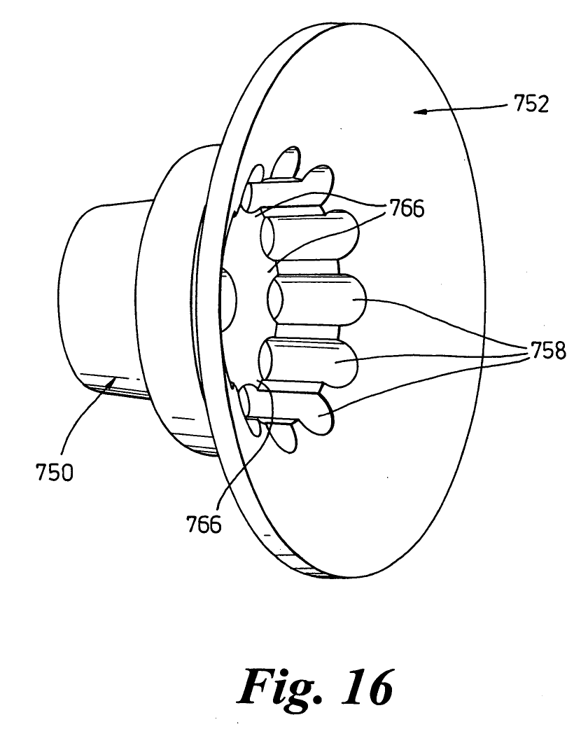

FIGS. 15 and 16 show a similar embodiment to that of FIGS. 13 and 14 in which the two parts 750 and 752 are coupled by narrow frangible sections or webs 766 instead of by two-shot moulding. The two parts may be produced as a single plastics moulding and webs 766 are defined by a circular array of openings 758 which surround the central part 750. A passageway 754 is also provided in the central part 750 and liquid ingress during filling takes place through the passageway 754 and the openings 758. Liquid egress however is restricted by surface tension effects to prevent glugging of the liquid out of the barrel 702 when the latter has been filled and is yet to be fitted with the needle injection unit.

As in the embodiment of FIGS. 13 and 14, forward movement of the plunger 706 is effective to release the various parts from one another by uncoupling the parts at the interfaces 426 and 742 and disrupting the narrow section webs 766 so that the central part 750 is freed to allow it together with the needle/hub assembly to be driven rearwardly into the hollow plunger 706 via the hole created by dislodging the blocking portion 738 from the rim 740.

In the embodiment of FIGS. 15 and 16, the outer part 752 is shown in the form of a conically-shaped disc with an uninterrupted outer periphery; however, if desired, its outer periphery may be provided with one or more cutaway portions as in the embodiment of FIGS. 13 and 14.

It will be appreciated that it is not intended to limit the invention to the above examples only, many variations, such as might readily occur to one skilled in the art, being possible, without departing from the scope thereof. Also certain features of the invention which are, for clarity, described in the context of separate embodiments, may also be provided in combination in a single embodiment. Conversely, various features of the invention which are, for brevity, described in the context of a single embodiment may also be provided separately or in any suitable sub-combination.

1. A hypodermic syringe comprising:
a barrel having an open end and provided with a restrictor in the vicinity of said open end,
a plunger slidable in the barrel for drawing liquid into and discharging liquid from the barrel through said open end, and
an injection needle unit for connection to the barrel, the restrictor having at least one aperture sufficiently small as ordinarily to prevent egress of liquid filling the barrel prior to connection of the injection needle unit to the barrel.

2. A syringe as claimed in claim 1 in which the needle unit includes an injection needle and a mounting portion for the needle and in which the restrictor is designed to allow passage of the needle mounting portion therethrough.

3. A hypodermic syringe having a barrel for use with a retractable needle assembly comprising a needle and a mounting hub for the needle, the barrel being provided with a restrictor in the vicinity of an open end of the barrel, the restrictor having at least one aperture which is sufficiently small as ordinarily to retain liquid within the barrel and being adapted to allow passage of the needle and mounting hub into the barrel.

4. A syringe as claimed in claim 3 in which the restrictor is located internally of the barrel at or adjacent an open end of the barrel.

5. A syringe as claimed in claim 3 in which a single aperture is provided therein, said aperture being sufficiently large to allow passage of the needle and hub therethrough.

6. A syringe as claimed in claim 3 in which the retractable needle assembly comprises a needle, a mounting hub therefor, a restrainer for holding the hub in a forward position and a retraction mechanism operable, upon release of the hub from the restrainer, to drive the needle and mounting hub rearwardly from said forward position into the barrel.

7. A syringe as claimed in claim 5 in which the plunger co-operates directly with the restrainer to effect release of the hub.

8. A syringe as claimed in claim 5 in which at or near the end of the forward stroke of the plunger, the forward end of the plunger contacts the restrictor so that forward movement of the plunger is transmitted through the restrictor to the restrainer in order to effect release of the hub.

9. A syringe as claimed in claim 3 in which the restrictor is suitable for use with liquids which have, at a temperature of 20 C, a fluid viscosity in the range of 0.6 to 70 cP and a surface tension in the range of 20 to 100 dynes/cm.

10. A syringe as claimed in claim 3 in which the barrel is provided with a hollow plunger and the retraction mechanism is operable to drive the needle into the hollow plunger.

11. A syringe as claimed in claim 3 in which the barrel is provided with a plunger having a forward end which approaches said open end of the barrel during discharge of liquid from the barrel and which has at its forward end a part which closes said forward end but is severable or dislodgeable from said forward end to allow the needle and mounting hub to be driven by the retraction mechanism into the hollow plunger.

12. A syringe as claimed in claim 3 in which the restrictor is a component separate from the barrel.

13. A syringe as claimed in claim 3 in which the restrictor is integral with the barrel.

14. A syringe as claimed in claim 3 in which the restrictor is deformable at least in the region of said aperture to allow passage of the needle and mounting hub therethrough.

15. A syringe as claimed in claim 3 in which the restrictor has or comprises a sealing formation which co-operates with part of the needle assembly whereby, during an injection stroke of the plunger, discharge of liquid from the needle assembly is confined to the pathway provided by the needle.

16. A syringe as claimed in claim 3 in which said aperture comprises a single hole provided with one or more inwardly directed projections for enhancing the ability of the restrictor to prevent egress of liquid from the barrel.

17. A syringe as claimed in claim 16, the projection or projections being capable of flexing.

18. A syringe as claimed in claim 3 in which said aperture of the restrictor is sized so that the hub and needle can pass therethrough during needle retraction.

19. A syringe as claimed in claim 3 in which said at least one aperture is of a size which is not sufficient to allow passage of the hub and needle.

20. A syringe as claimed in claim 3 in which the restrictor includes a portion which can be removed to allow passage of the needle and hub during the needle retraction process.

21. A syringe as claimed in claim 3 in which the restrictor is capable of being disrupted in such a way that it no longer blocks the needle and hub when needle retraction is required.

22. A syringe as claimed in claim 3 in which the restrictor comprises an inner portion and an outer portion, the inner portion being severable or otherwise releasable from the outer portion to create an opening sufficiently large to allow passage of the needle and hub during needle retraction.

23. A syringe as claimed in claim 22 in which the inner and outer portions are demarcated from one another by a zone or line of weakness at which the inner portion is severable from the outer portion during needle retraction.

24. A syringe as claimed in claim 22 in which the zone or line of weakness may be provided by one or more webs of material interconnecting the two portions.

25. A syringe as claimed in claim 22 in which the inner and outer portions are interconnected at a moulding interface therebetween such that the inner portion is severed or dislodged from the outer portion as the plunger approaches or reaches completion of its forward stroke.

26. A syringe as claimed in claim 22 in which the inner portion is provided with said at least one aperture.

27. A syringe as claimed in claim 22 in which the inner portion is of tubular configuration defining a passageway constituting said aperture.

28. A syringe as claimed in claim 3 in which the restrictor comprises a disc located within the barrel.

29. A syringe as claimed in claim 28 in which, when in situ within the barrel, the disc is of generally conical configuration with said aperture at its apex.

30. A syringe as claimed in claim 28 in which the disc is self-retaining in the barrel once inserted.

31. A syringe as claimed in claim 3 in which the restrictor comprises an annular flange projecting inwardly of the barrel, the flange defining said aperture.

32. A syringe as claimed in claim 31 in which the flange is resiliently deflectable radially outwardly.

33. A syringe as claimed in claim 31 in which the flange is formed integrally with the barrel adjacent its open end.

34. A syringe as claimed in claim 31 in which the flange acts as a lip seal by co-operation with a component of the needle assembly.

35. A syringe as claimed in claim 3 in which the restrictor is perforated or reticulated to prevent glugging of the liquid out of the barrel.

36. A syringe as claimed in claim 3 in which the restrictor presents an array of apertures distributed over the cross-sectional area of the barrel.

37. A syringe as claimed in claim 3 in which the restrictor is of a frangible or brittle material or rendered frangible or brittle in a defined zone or zones thereof so that, during forward movement of the plunger, the integrity of the restrictor is disrupted to allow passage of the hub/needle assembly during needle retraction.

38. A syringe as claimed in claim 3 in which the restrictor comprises an inner part and an outer part which are normally coupled together but which are released from one another during or just prior to needle retraction.

39. A syringe as claimed in claim 38 in which the outer part is restrained against forward movement or only allowed to move forwardly to a limited extent relative to the barrel.

40. A syringe as claimed in claim 38 in which, after release of the inner part from the outer part, the inner part leaves an opening in the restrictor sufficiently large to allow passage of the needle/hub assembly during needle retraction.

41. A syringe as claimed in claim 38 in which the inner part is provided with a central passageway for liquid flow into and out of the barrel, the passageway being sufficiently small as ordinarily to retain liquid within the barrel when pressure is not being applied to the plunger to move it forwardly.

42. A syringe as claimed in claim 38 in which the two parts of the restrictor are coupled together by virtue of one part having been moulded in the presence of the other part.

43. A syringe as claimed in claim 38 in which the two parts are coupled together via frangible sections of material which can readily break to allow release of the inner part from the outer part when required.

44. A syringe as claimed in claim 3 in which the arrangement is such that the needle unit is engaged with the barrel through a one-way coupling means formed by interfitting components on the needle unit and the barrel whereby the injection needle once assembled to the barrel cannot be disengaged from the barrel.

45. A barrel for use with a retractable needle assembly comprising a needle and a mounting hub for the needle, the barrel being provided with a restrictor located internally of the barrel at or adjacent an open end of the barrel, the restrictor having at least one aperture which is sufficiently small as ordinarily to retain liquid within the barrel and being adapted to allow passage of the needle and mounting hub into the barrel.
